(19) United States
(12) Patent Application Publication
(10) Pub. No.: US 2025/0232844 A1
(43) Pub. Date: Jul. 17, 2025

(54) Automated, Configurable, Rigorous Reversible Lumping for Chemical Separations

(71) Applicant: AspenTech Corporation, Bedford, MA (US)

(72) Inventors: Zhen Hou, Lexington, MA (US); Lingxiang Li, Shanghai (CN); Lili Yu, Shanghai (CN); Shu Wang, Lexington, MA (US); Darin Campbell, Waltham, MA (US)

(21) Appl. No.: 18/853,912

(22) PCT Filed: Apr. 7, 2022
(86) PCT No.: PCT/CN2022/085499

§ 371 Date: Oct. 3, 2024

(51) Int. Cl. G16C 20/10; G05B 13/04; G16C 20/70

(52) U.S. Cl. CPC G16C 20/10; G05B 13/04; G16C 20/70

(57) ABSTRACT

Described is a computer-implemented method for modeling an equilibrium separation in a chemical separator. The method can include representing a feedstock of the chemical separator as a collection of molecules, each molecule having a mole fraction. A cluster analysis is performed on the feedstock based on a property of the collection of molecules to generate thermodynamic lumps. A mapping identity table is generated that identifies each molecule of the collection of molecules in the feedstock. A simulation of a chemical separation of the thermodynamic lumps is performed. The mole fraction of molecules in a resultant first phase and the mole fraction of molecules in a resultant second phase is determined.

### BACKGROUND

Existing computer-based methods and systems for modeling chemical reactions can model thousands of species and similarly, thousands of reactions. In particular, molecular modeling provides an optimal solution for refineries to simulate crude to chemical (CTOC) scenarios. Molecule-Based (MB) Equation Oriented Reactor and Molecular Characterization models simulate conversion units and feed characterization at the molecular level. In addition, separation units are also important to be considered at molecular level in a flowsheet of refinery-wide models such as CTOC. Propagation of molecular information between MB reactors and separation processes is a foundation for a multi-units flowsheet simulation such as CTOC at the molecular level.

### SUMMARY

A MB reactor model may contain in the order of 10,000 species to describe the molecular details of the reactions. However, it is a numerical challenge to apply certain separation models to such a large number of species. Accordingly, there is a need for improved computer-implemented methods and systems for modeling chemical reactions

Embodiments of the present invention provide methods, systems, and computer program products for modeling an equilibrium separation in a chemical separator. Embodiments can determine the mole fraction of molecules in the resultant first phase and the mole fraction of molecules in the resultant second phase. Embodiments can control a separation process based on a determined mole fraction of molecules in a resultant first phase and/or a determined mole fraction of molecules in a resultant second phase. The methods, systems, and computer program products described herein reduce the computational burden when modeling a chemical separation.

One embodiment involves representing a feedstock of the chemical separator as a collection of molecules, each molecule having a mole fraction. A cluster analysis is performed on a property, or in some embodiments a combination of properties, of molecules of the collection of molecules to generate thermodynamic lumps. A mapping identity table is generated that identifies each molecule of the collection of molecules in the feedstock. A simulation of a chemical separation of the thermodynamic lumps is performed to generate a mole fraction of each thermodynamic lump in a resultant first phase and a mole fraction of each thermodynamic lump in a resultant second phase. The mole fraction of molecules in the resultant first phase and the mole fraction of molecules in the resultant second phase is determined based on the mapping identity table, the mole fraction of each thermodynamic lump in the resultant first phase, and the mole fraction of each thermodynamic lump in the resultant second phase.

In embodiments, the steps of the method, i.e., the representing, performing, generating, performing, and determining, may be automatically performed or may be performed responsive to user input.

In some embodiments, the feedstock can be an unrefined chemical composition, a hydrocarbon mixture, biomass, shale oil, plastic, lignin, cellulose, or any other feedstock to be separated.

The property of molecules of the collection of molecules can be a thermodynamic property, such as a Kᵢ criteria. Examples of Kᵢ criteria are boiling point, vapor pressure, a solubility parameter, melting point, and enthalpy of fusion (ΔH_{fus}). The property can also be one or more structural attributes of the molecules of the collection of molecules, such as: i) compound class; and ii) number of carbon atoms. The compound class can include one or more of aromatic, naphthenic, isoparaffin, paraffin, and olefin.

The cluster analysis can be K-Mean method AS136, K-Mean method AS58, or Ward's minimum variance method. The method can include receiving user input selecting the cluster analysis. The method can include receiving user input selecting a total number of thermodynamic lumps. The method can include receiving user input selecting a maximum number of molecular species in the thermodynamic lumps. The method can include receiving user input selecting particular molecules from the collection of molecules for a thermodynamic lump. The method can include receiving user input selecting particular molecules from the collection of molecules that are excluded from a thermodynamic lump.

The variety of equilibrium processes can be modeled. In a vapor-liquid equilibrium (VLE), the resultant first phase can be a vapor phase and the resultant second phase can be a liquid phase. In a liquid-liquid equilibrium (LLE), the resultant first phase is a liquid phase and the resultant second phase is a liquid phase. In a solid-liquid equilibrium (SLE), the resultant first phase is a solid phase and the resultant second phase is a liquid phase.

The method can further include controlling a separation process based on one or more of the mole fraction of molecules in the resultant first phase and the mole fraction of molecules in the resultant second phase.

Another embodiment is directed to a system for performing the methods described herein. The system includes a processor and a memory with computer code instructions stored thereon. The processor and the memory, with the computer code instructions, being configured to cause the system to perform the methods described herein.

Yet another embodiment is directed to a computer program product for performing the methods described herein. The computer program product includes a computer readable medium with computer code instructions stored thereon where the computer code instructions, when executed by a processor, cause an apparatus associated with the processor to perform any embodiments described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a depiction of a flowsheet of a commercial hydrocracker that embodiments described herein can be used to model.

FIG. 2 is a flowchart depicting a method for modeling an equilibrium separation in a chemical separator according to an embodiment.

FIG. 3 is a representation of a simple vapor-liquid equilibrium (VLE) flash example that may be used in embodiments.

FIG. 4 is a representation of a workflow of an Automated Configurable Rigorous Reversible Lumping (ACRRL) technique according to embodiments.

FIG. 5 is a parity plot that illustrates the comparison of results of the process in the vapor phase of a flash product simulated by embodiments.

FIG. 6 is a parity plot that illustrates the comparison of results of the process in the liquid phase of a flash product simulated by embodiments.

FIG. 7 is a plot showing the curves of vapor-liquid distribution ratio (Kᵢ) values versus boiling point for a molecular based full flash model and molecular based lumped flash model simulated by embodiments.

FIG. 8 is a flowsheet representation of a system where embodiments may be employed.

FIG. 9 is a plot comparing results of heavy oil precipitation in terms of different number of liquid-liquid equilibrium lumps.

FIG. 10 is a plot comparing an asphaltene precipitation curve simulated by embodiments with an asphaltene precipitation curve from literature.

FIG. 11 depicts a computer network or similar digital processing environment in which embodiments of the present invention may be implemented.

FIG. 12 is a diagram of an example internal structure of a computer in the environment of FIG. 11.

### DETAILED DESCRIPTION

A description of example embodiments follows.

The teachings of all patents, published applications and references cited herein are incorporated by reference in their entirety.

### Overview

Existing methods for simulating chemical reactions, such as Aspen Technology, Inc.'s Molecule-Based EO (Equation Oriented) Reactor (MB EORXR) described in U.S. patent application Ser. No. 16/250,445, published as US 2019/0228843 A1, allow users to model refining chemistries at the molecular level. MB EORXR can use more than 10,000 species and 5700 reactions to describe the conversion of hydrocarbon mixtures up to and including resid. However, the structures and reactions of heavy resid modeled in the molecule based hydrocracker/hydrotreater of MB EORXR are still limited. According to recent analytical chemistry research, there are hundreds of distinct aggregated ring structures in the heavy petroleum resid fraction that determine the reactivity, thermodynamics, and key properties of petroleum. Based on this research, there are millions of individual heavy molecular structures. Computational resources are a significant challenge to model such a large system via existing methods, such as MB EORXR. The statistics of the computational requirements for a molecule based reactor that models full detailed compositions from naphtha through heavy resid (referred to as “full MB model” herein) are listed in Table 1.

TABLE 1
Statistics of computational requirements for MB EO
reactor (O = “in the order of’)

| | Naphtha | Diesel | Gasoil | VGO | Resid |
|---|---|---|---|---|---|
| # of species | O(200) | O(500) | O(1000) | O(2000) | O(>10\hat{ }6) |
| # of reactions | O(500) | O(1000) | O(2500) | O(5000) | O(>10\hat{ }6) |
| # of Equation in | O(3*10\hat{ }4) | O(6*10\hat{ }4) | O(2*10\hat{ }5) | O(3*10\hat{ }5) | O(>10\hat{ }7) |
| a single bed | | | | | |
| # of Equation in a | O(2*10\hat{ }5) | O(5*10\hat{ }5) | O(10\hat{ }6) | O(>10\hat{ }6) | O(>10\hat{ }8) |
| hydrocracker (4 beds) | | | | | |

Table 1 shows that the number of molecular components and reactions increases exponentially from light naphtha to heavy resid. As a result, the number of equations required to model a reactor bed also grows dramatically from naphtha to resid. Furthermore, modelling a complex flowsheet including 2-10 reactor beds requires even more computational resources. For instance, the number of equations and variables for a 4 bed hydrocracker is almost one order of magnitude larger than that of a single reactor bed. The large number of equations needed to perform these simulations can significantly affect the computational performance of an equation oriented model.

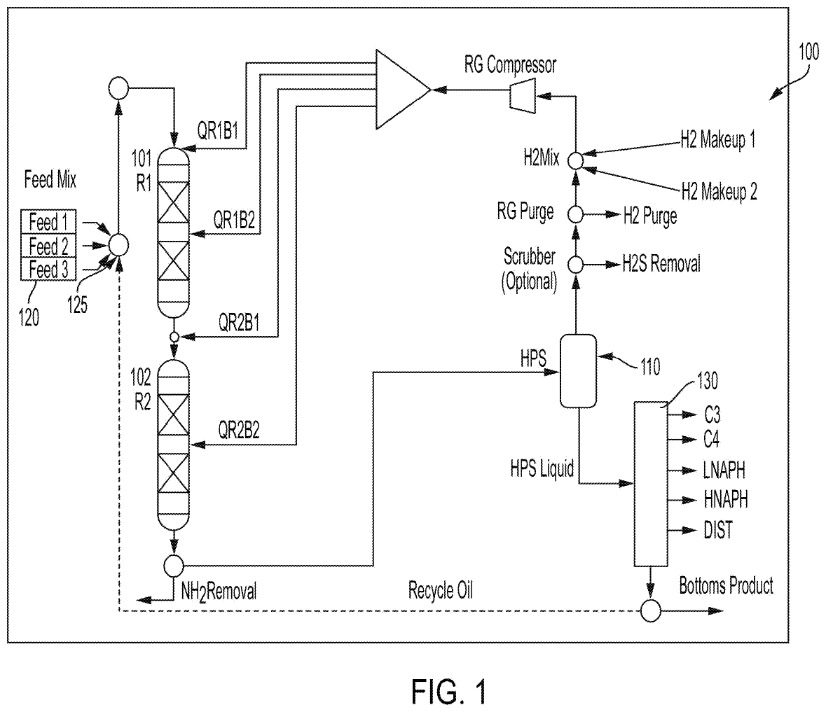

FIG. 1 shows a typical flowsheet of a commercial hydrocracker (HCR) 100. The two reactors 101 and 102, each having two reactor beds, are created by a reactor simulator. In addition to the reactor blocks, there is a set of necessary flowsheet blocks required to build a hydrocracker flowsheet, such as feed blocks for intakes 120 and a feed mixer 125. Some blocks, such as the flash for the High Pressure Separator (HPS) 110, the product fractionator 130, which can be a short-cut column or a rigorous column, require a complex process model based on a rigorous thermodynamic vapor-liquid equilibrium (VLE) model. Moreover, the extractor units (e.g., de-asphalter) also coexist in a flowsheet of refinery-wide models and thus need to be modeled by rigorous thermodynamic or short-cut liquid-liquid equilibrium (LLE) models. Therefore, the propagation of molecular information between MB reactor and VLE or LLE based separation process is a foundation for a multi-units flowsheet simulation such as CTOC at molecular level.

The MB reactor model contains in the order of 10,000 species to describe the molecular details of the reactions. However, it is a numerical challenge to apply VLE and LLE models to such a large number of species. The binary coefficients of thermodynamic models can lead the number of variables used in a single thermodynamic model to be in the order 10⁸ (i.e., 10,000*10,000). The computational resource requirements of such a large model make it impractical to solve multi-units flowsheet simulations. In addition, the approximately 10,000 species can contain the molecular compositions ranging from naphtha to resid. The molecular components in the heavier fractions (e.g., resid) often have large carbon numbers, aggregated aromatic rings and multiple heteroatoms. Due to the lack of experimental data, it is challenging to obtain accurate thermodynamic properties of those complex components

To utilize a molecular-level reactor model, the reactor models are able to connect to the flowsheet of a refinery. An example of a flow sheet engine used in chemical process simulators is Aspen HYSYS Petroleum Refining (Aspen HPR), used in Aspen HYSYS. Aspen HYSYS is a simulation software package that can be used to model refinery and chemical plants offered by Aspen Technology, Inc. The assay-based components defined in a flowsheet engine used in the reactor models are essentially VLE driven hypothetical (hypo) components. Since in the order of 10,000 molecules is too large to model VLE calculations, it is necessary to develop an approach that can propagate the molecular details of the MB reactor model across the entire flowsheet by mapping the in the order of 10,000 molecules to a much smaller number of hypothetical components.

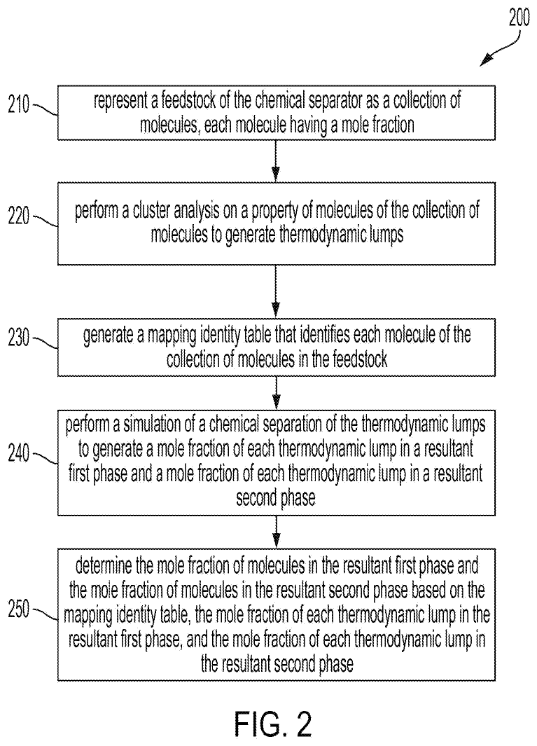

Therefore, a new approach is needed to reduce the computational burden of a large MB EO reactor model and maintain the robustness of the model solution. FIG. 2 illustrates one such example method 200. The method 200 is computer implemented and may be performed via any combination of hardware and software as is known in the art. For example, the method 200 may be implemented via one or more processors with associated memory storing computer code instructions that cause the processor to implement steps 210, 220, 230, 240, and 250 of the method 200. Further, the method 200 may be implemented in conjunction with existing simulation software, such as Aspen Technology, Inc.'s Molecule-Based EO (Equation Oriented) Reactor (MB EORXR), described in U.S. patent application Ser. No. 16/250,445, published as US 2019/0228843 A1. In such an implementation, aspects of method 200 and/or any other embodiments described herein may be implemented in blocks generated by MB Reactor Builder.

The method 200 begins at step 210 by representing, representing a feedstock of the chemical separator as a collection of molecules, each molecule having a mole fraction.

The collection of molecules can be represented in a variety of ways. In one embodiment, the collection of molecules is an index or list that relates a plurality of molecules to a unique identifies. In another embodiment, the collection of molecules is individual molecule representations and molecular attribute representations, as disclosed in U.S. application Ser. No. 16/739,291, published as US 2021/0217497 A1. The latter embodiment provides additional benefits because it further reduces computing requirements.

The feedstock may be an unrefined chemical composition, a hydrocarbon mixture, biomass, shale oil, plastic, lignin, cellulose, or any other feedstock to be separated.

The method 200 continues and at step 220 by performing a cluster analysis on a property of the collection of molecules to generate thermodynamic lumps. Each thermodynamic lump can have a maximum number of molecular species. The cluster analysis algorithm is used to determine the number of thermodynamic lumps of that separation process. The property can be a thermodynamic property, such as a Kᵢ criteria. Examples of Kᵢ criteria are boiling point, vapor pressure, a solubility parameter, and melting point. The property can be a combination of: i) compound class; and ii) number of carbon atoms. The compound class can include one or more of aromatic, naphthenic, isoparaffin, paraffin, and olefin. The criteria of the cluster analysis are dependent on the nature of phase equilibrium of that separation process (e.g., a criteria pertaining to distribution between phases) and usually are one or more properties of the molecules themselves.

The cluster analysis can be K-Mean method AS136, K-Mean method AS58, or Ward's minimum variance method. The default cluster analysis is K-Mean method AS136. A user can provide input to select the cluster analysis, select a total number of thermodynamic lumps, select the maximum number of molecular species in the thermodynamic lumps, select particular molecules from the collection of molecules for a thermodynamic lump, or select particular molecules from the collection of molecules that are excluded from a thermodynamic lump. Specifying the details of the thermodynamic lumps allows a user to fine-tune the granularity of the lumps for a particular application or separation process.

At step 230, the method generates a mapping identity table that identifies each molecule of the collection of molecules in the feedstock. The identity mapping table is used in step 250 to determine the mole fraction of molecules in the resultant first phase and the mole fraction of molecules in the resultant second phase.

At step 240, the method performs a simulation of a chemical separation of the thermodynamic lumps to generate a mole fraction of each thermodynamic lump in a resultant first phase and a mole fraction of each thermodynamic lump in a resultant second phase.

At step 250, a simulation of a chemical separation process is performed using the limited number of thermodynamic lumps, the simulation determining composition of the products of the separation process.

The method 200 may also perform further processing or take real-world actions based upon the mole fraction of molecules in the resultant first phase and the mole fraction of molecules in the resultant second phase, as determined in step 250.

A simulation of a chemical separation of the thermodynamic lumps to generate a mole fraction of each thermodynamic lump in a resultant first phase and a mole fraction of each thermodynamic lump in a resultant second phase is performed. The simulation can be performed by existing simulation blocks, such as those available in HYSYS and/or AspenPlus. For example, the flash, column, and extractor block simulations can be performed.

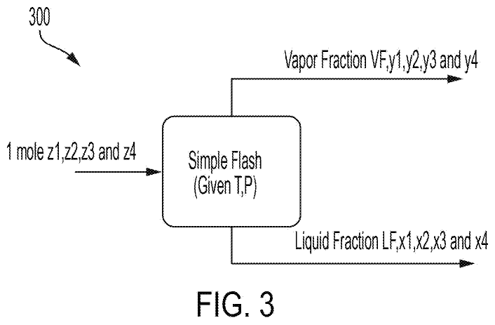

Aspen HYSYS and Aspen Plus are simulation software packages that can be used to model refinery and chemical plants. While example embodiments may be described in connection with the Aspen HYSYS or Aspen Plus, it will be understood by those skilled in the art that various changes in form and details may be made therein without departing from the scope of the embodiments encompassed by the appended claims. In the convention of Aspen HYSYS and Aspen Plus, separation process models (e.g., VLE blocks, LLE blocks) are core components for units such as, separators, columns, etc. As noted previously, there is a computational resource limitation for modeling in the order of 10,000 molecular species in Aspen separation blocks. These embodiments propose a novel approach to reduce the number of species in separation blocks while keeping track of the molecular profile of in the order of 10,000 molecular species, via grouping to a limited number of thermodynamic lumps to complete VLE or LLE based separation calculations in model process modeling software packages, such as Aspen HYSYS and Aspen Plus. The grouping of the molecular species to thermodynamic lumps is a significant challenge to maintain the molecular profile in those separation calculations. To keep the molecular information during the calculations of separation processes, the essential calculation criterion of the VLE or LLE model need to be determined. For example, consider a simple VLE flash example shown as FIG. 3.

### Example Implementation

In FIG. 3, there are four components in a simple T-P flash 300. The mole fractions of the inlet feed are marked as z₁, z₂, z₃ and z₄. The mole fractions of the vapor phase are marked as y₁, y₂, y₃ and y₄; and the mole fractions of the liquid phase are marked as x₁, x₂, x₃ and x₄. The vapor fraction of the product is V_{F} and the liquid fraction of the product is L_{F}.

In general, the equilibrium relationship of a given component i between vapor phase and liquid phase is shown in Eq. 1.

$${K}_{i} = \frac{{y}_{i}}{{x}_{i}} \qquad Eq . 1$$

Kᵢ is vapor-liquid distribution ratio
yᵢ is the fraction of component in the vapor phase
xᵢ is the fraction of component in the liquid phase

Using the Rachford-Rice method to solve this problem, the equations can be shown as Eq. 2 to Eq. 6.

$${V}_{F} = 1 - {L}_{F} \qquad Eq . 2$$

$${y}_{1} = {K}_{1} {x}_{1} ; {y}_{2} = {K}_{2} {x}_{2} ; {y}_{3} = {K}_{3} {x}_{3} ; {y}_{4} = {K}_{4} {x}_{4} \qquad Eq . 3$$

$${y}_{1} = \frac{{K}_{1} {z}_{1}}{1 + {V}_{F} ( {K}_{1} - 1 )} ; {y}_{2} = \frac{{K}_{2} {z}_{2}}{1 + {V}_{F} ( {K}_{2} - 1 )} ; {y}_{3} = \frac{{K}_{3} {z}_{3}}{1 + {V}_{F} ( {K}_{3} - 1 )} ; {y}_{4} = \frac{{K}_{4} {z}_{4}}{1 + {V}_{F} ( {K}_{4} - 1 )} \qquad Eq . 4$$

$${x}_{1} = \frac{{z}_{1}}{1 + {V}_{F} ( {K}_{1} - 1 )} ; {x}_{2} = \frac{{z}_{2}}{1 + {V}_{F} ( {K}_{2} - 1 )} ; {x}_{3} = \frac{{z}_{3}}{1 + {V}_{F} ( {K}_{3} - 1 )} ; {x}_{4} = \frac{{z}_{4}}{1 + {V}_{F} ( {K}_{4} - 1 )} \qquad Eq . 5$$

$$0 = \frac{{z}_{1} ( {K}_{1} - 1 )}{1 + {V}_{F} ( {K}_{1} - 1 )} + \frac{{z}_{2} ( {K}_{2} - 1 )}{1 + {V}_{F} ( {K}_{2} - 1 )} + \frac{{z}_{3} ( {K}_{3} - 1 )}{1 + {V}_{F} ( {K}_{3} - 1 )} + \frac{{z}_{4} ( {K}_{4} - 1 )}{1 + {V}_{F} ( {K}_{4} - 1 )} \qquad Eq . 6$$

Suppose component1 and component2 have the same K value and component3 and component4 have the same K value as shown in Eq. 7. The four components can be grouped by K values as shown in Eq. 8 to Eq. 12 and then use the grouped variables (X̅₁, Y̅₁, Z̅₁ for component 1 and 2; X̅₂, Y̅₂, Z̅₂ for component 3 and 4) to resolve the problem by the Rachford-Rice method as shown with Eq. 13 to Eq. 15. As a result, the mole fractions of individual component 1-4 can be calculated by Eq. 16 and Eq. 17 for the vapor and liquid phases respectively.

$${K}_{1} = {K}_{2} ; {K}_{3} = {K}_{4} \qquad Eq . 7$$

$${y}_{1} = {K}_{1} {x}_{1} ; {y}_{2} = {K}_{2} {x}_{2} ; {y}_{3} = {K}_{3} {x}_{3} ; {y}_{4} = {K}_{4} {x}_{4} \qquad Eq . 8$$

$$\frac{{y}_{1}}{{y}_{2}} = \frac{{z}_{1}}{{z}_{2}} = \frac{{x}_{1}}{{x}_{2}} ; \frac{{y}_{3}}{{y}_{4}} = \frac{{z}_{3}}{{z}_{4}} = \frac{{x}_{3}}{{x}_{4}} \qquad Eq . 9$$

$${f}_{11} = \frac{{z}_{1}}{{z}_{1} + {z}_{2}} ; {f}_{12} = \frac{{z}_{2}}{{z}_{1} + {z}_{2}} ; {f}_{21} = \frac{{z}_{3}}{{z}_{3} + {z}_{4}} ; {f}_{22} = \frac{{z}_{4}}{{z}_{3} + {z}_{4}} \qquad Eq . 10$$

$${\overline{X}}_{1} = {x}_{1} + {x}_{2} ; {\overline{X}}_{2} = {x}_{3} + {x}_{4} ; {\overline{Y}}_{1} = {y}_{1} + {y}_{2} ; {\overline{Y}}_{2} = {y}_{3} + {y}_{4} ; \qquad Eq . 11$$

$${\overline{Z}}_{1} = {z}_{1} + {z}_{2} ; {\overline{Z}}_{2} = {z}_{3} + {z}_{4} \qquad Eq . 12$$

$$0 = \frac{{\overline{Z}}_{1} ( {K}_{1} - 1 )}{1 + {V}_{F} ( {K}_{1} - 1 )} + \frac{{\overline{Z}}_{2} ( {K}_{3} - 1 )}{1 + {V}_{F} ( {K}_{3} - 1 )} \qquad Eq . 13$$

$${\overline{Y}}_{1} = \frac{{K}_{1} {\overline{Z}}_{1}}{1 + {V}_{F} ( {K}_{1} - 1 )} ; {\overline{Y}}_{2} = \frac{{K}_{3} {\overline{Z}}_{2}}{1 + {V}_{F} ( {K}_{3} - 1 )} \qquad Eq . 14$$

$${\overline{X}}_{1} = \frac{{\overline{Z}}_{1}}{1 + {V}_{F} ( {K}_{1} - 1 )} ; {\overline{X}}_{2} = \frac{{\overline{Z}}_{2}}{1 + {V}_{F} ( {K}_{3} - 1 )} \qquad Eq . 15$$

$${y}_{1} = {f}_{11} {\overline{Y}}_{1} ; {y}_{2} = {f}_{12} {\overline{Y}}_{1} ; {y}_{3} = {f}_{21} {\overline{Y}}_{2} ; {y}_{4} = {f}_{22} {\overline{Y}}_{2} \qquad Eq . 16$$

$${x}_{1} = {f}_{11} {\overline{X}}_{1} ; {x}_{2} = {f}_{12} {X}_{1} ; {x}_{3} = {f}_{21} {\overline{X}}_{2} ; {x}_{4} = {f}_{22} {\overline{X}}_{2} \qquad Eq . 17$$

Therefore, if the molecular species that have the same value of Kᵢ are lumped together as one single VLE hypo, the VLE behavior of those molecular species are the same and the internal molecular distribution fᵢⱼ for a given lump hypo i is constant before and after the VLE model. As a result, the molecular composition of a given lump can be rigorously and reversibly estimated back from a lump following the logic in Eq. 16 and Eq. 17.

The above approach may also be applied to a LLE problem by altering the variables of vapor phase/liquid phase to the variables of light liquid phase/heavy liquid phase in Eq. 1 to Eq. 17. And Kᵢ is light liquid-heavy liquid distribution ratio.

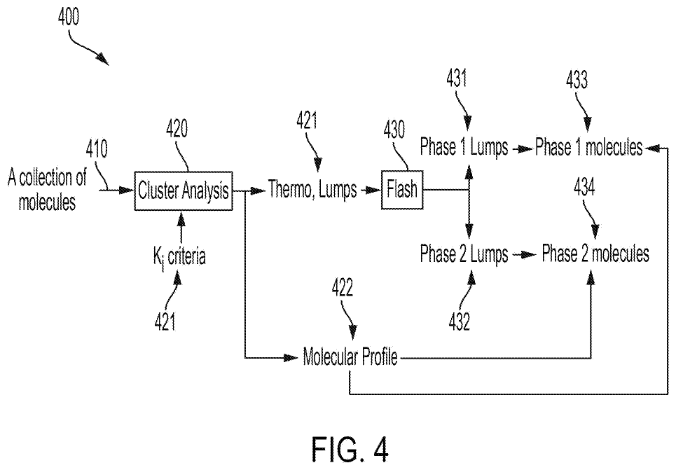

Therefore, Kᵢ is a significant criterion of general thermodynamic phase equilibrium calculations. However, the Kᵢ of components for a large scale system is not an intuitive physical property that can be used to lump the molecular compositions. Therefore, there is a need to find apparent properties as the criteria. Moreover, the apparent properties to determine various phase equilibrium problems (e.g., VLE, LLE) are different. To provide a general solution for a wide range of phase equilibrium process models, an Automated Configurable Rigorous Reversible Lumping (ACRRL) technique is described. The conceptual workflow 400 of ACRRL is shown in FIG. 4.

Starting from a collection of molecules in the feed stream 410 of a given separation process, ACRRL executes a cluster analysis algorithm 420 based on a property, such as a thermodynamic property (e.g., a Kᵢ criteria), to determine the size of thermodynamic lumps of that separation process. The criteria of the cluster analysis are dependent on the nature of phase equilibrium of that separation process and usually are one or more properties of the molecules themselves (e.g., boiling point, vapor pressure, solubility parameter, and melting point). In some embodiments, the lumping criteria is a combination of compound class (e.g., aromatic, naphthenic, isoparaffin, paraffin, and olefin) and number of carbon atoms. The default cluster analysis method in ACRRL is K-Mean method AS136. See generally J. A. Hartigan and M. A. Wong, “A K-Means Clustering Method,” J. Roy. Stat. Soc., Series C (Applied Statistics) Vol. 28, No. 1, 100-108 (1979). In some embodiments, users also can select K-Mean method AS58. See generally, D. N. Sparks, “Algorithm AS 58: Euclidean Cluster Analysis,” J. Roy. Stat. Soc., Series C (Applied Statistics), Vol. 22, No. 1, 126-130 (1973). In some embodiments, the cluster analysis method is Ward's minimum variance method. See generally, Ward, J. H., Jr., “Hierarchical Grouping to Optimize an Objective Function,” J. Am Stat. Assoc., 58, 236-244 (1963). The number of thermodynamic lumps can be specified by the user in order to adjust the granularity of thermodynamic lumps. Often, individual small molecules do not need to be defined by lumps. The accuracy of separation results of those small molecules can be important for industrial practice (e.g., debutanizer in FCC). ACRRL provides a flexible way to handle those isomers without lumping by specifying some structural configurations such as one or more of an explicit molecule list, compound class (e.g., aromatic, naphthenic, isoparaffin, paraffin, and olefin), and carbon number range. This option allows users to keep important individual molecules in separation processes without lumping. Further, by setting the number of clusters to be equal to the number of molecules, ACRRL can push all individual molecular compositions to separation blocks without any lumping. Therefore, the cluster analysis in ACRRL is not only able to reduce a large number of molecules to a smaller number of thermodynamic lumps but also maintain selected individual isomers for a given separation process.

After the cluster analysis, ACRRL transfers the full molecular details of the feed stream to thermodynamic lumps 421 and stores the internal molecular profile 422 of the molecules in the given thermodynamic lumps. Then, those thermodynamic lumps 421 are sent to a flash separation model 430 to calculate phase equilibrium and simulate the separation process. As one example, the flash block 430 may be the flash block 300 described with respect to FIG. 3 to represent that separation process. The effluents of that flash block are sets of thermodynamic lumps for different phases (e.g., phase 1 lumps 431 and phase 2 lumps 432 in FIG. 4).

Because the internal molecular profile of a given lump is kept during the separation calculation following the approach illustrated in Eq. 1 to Eq. 17, Eq. 16 and Eq. 17 can be used to map the detailed molecular compositions of first resultant phase 433 and the second resultant phase 434 from the thermodynamic lumps of outlet streams and the internal molecular profiles 422 of those thermodynamic lumps.

Consequently, the separation models of molecular streams within the order of 10,000 molecules can be simulated with a limited number of thermodynamic lumps but maintaining the full molecular details in the products via ACRRL.

The ACRRL is implemented as two functional blocks: ACRRL Lumper and ACRRL De-Lumper. The ACRRL Lumper is used to lump molecular compositions in the MB reactor model into Aspen thermodynamic lumps following ACRRL rules.

The first step of the ACRRL Lumper is to build a mapping identity table between thermodynamic lumps and molecular compositions shown in Eq. 18.

$$[ \begin{matrix}{SpcIndex}_{11} & ⋯ & {SpcIndex}_{1 p} \\ {SpcIndex}_{12} & ⋯ & {SpcIndex}_{1 p} \\ ⋯ & ⋯ & ⋯ \\ & {SpcIndex}_{ij} & \\ ⋯ & ⋯ & ⋯ \\ ⋯ & & ⋯ \\ {SpcIndex}_{m 1} & ⋯ & {SpcIndex}_{mp}\end{matrix} ] \qquad Eq . 18$$

All molecular species can be lumped into m thermodynamic lumps from 1 to m. p is the maximum number of molecular species lumped in a thermodynamic lump by counting all of the molecular species in all the lumps. A table of dimension m*p is created to store the identities of the full molecular species. If the total number of molecular species is n, an arbitrary index of each species can be assigned and a vector [1 . . . n] may be formulated to represent the identities of n molecular species by arbitrary indices. Using the cluster analysis, [1 . . . n] of species indices can be mapped to the table in Eq. 18. SpcIndexᵢⱼ is referred to as the molecular species of the jth element in thermodynamics lump i and ip is the actual number of the molecular species in that lump i. ip is always <=p. The value of SpcIndexᵢⱼ is the index value of this species in the vector [1 . . . n]. Since one species can only be mapped into one row of the table in Eq. 18, the total number of SpcIndexᵢⱼ is equal to the total number of molecular species.

After constructing the mapping identity table in Eq. 18, the next step is to calculate the value of each thermodynamics lump. The input of this calculation is a vector of mole fractions of n molecular species: ymol[1 . . . n]. The output is a vector of mole fractions of m thermodynamics lumps: ylump[1 . . . m]. The equation to obtain the mole fractions in ylump is shown in Eq. 19.

$$ylump [ i ] = {\sum }_{j = 1}^{ip} ymol [ {SpcIndex}_{ij} ] when {SpcIndex}_{ij} \geq 0 \qquad Eq . 19$$

In addition to obtaining ylump, the internal molecular profile of a given lump i needs to be calculated. To save the memory cost of the model, a mapping vector is created: ymap[1 . . . n] to store the values in the molecular profiles for all thermodynamic lumps. The calculation step is a straightforward normalization as shown in Eq. 20.

$$ymap [ {SpcIndex}_{i j} ] = \frac{y m o l [ SpcInde {x}_{ij} ]}{ylum p [ i ]} when {SpcIndex}_{i j} \geq 0 \qquad Eq . 20$$

Eq. 18 is a pre-processing function of the ACRRL Lumper. The values of Eq. 18 are not counted as variables in equation-oriented Aspen Plus (Aspen EO). Eq. 19 and Eq. 20 are written in terms of the Aspen EO format including residuals, sparsity patterns and analytical jacobians. The number of equations in ACRRL Lumper is equal to m+n. The size of this block is moderate and thus does not significantly affect the performance of the MB model.

The properties of the thermodynamic lumps are derived from in the order of 10,000 molecular species in the model. All properties or relevant parameters of all molecules are predefined in the MB framework from literature, the Aspen thermodynamic database, and subject matter expertise. The relationship between a given thermodynamic lump and a set of molecules is explicitly defined in the identity mapping table Eq. 18. So, the properties of a given lump i is calculated from the molecules in the ith row of Eq. 18. The linear mixing rules can be applied to estimate most of structural properties such as carbon number, molecular weight, aromatic ring number etc. and some thermodynamic properties: standard formation of enthalpy, standard formation of entropy, etc. Other thermodynamic properties such as boiling point, critical properties can be calculated by alternative methods. For example, if it is assumed that boiling point is estimated from a detailed group contribution method for a large molecule, the linear mixing rule can be applied to calculate the functional groups in a given lump from those functional groups of the molecules allocated to that lump and calculate the value of the boiling point of that lump from the estimated functional groups of that lump. More detailed methods to estimate thermodynamic phase change properties/parameters based on users' expertise can easily be integrated with Aspen comprehensive property package via inp files if users need to use Aspen comprehensive property package to perform their separation calculations.

After separation calculations, mole fractions of the thermodynamic lumps need to be transferred back to the mole fractions of the full molecular species in order to propagate the molecular information to the next MB model block. The ACRRL De-Lumper block is implemented for this purpose. The ACRRL De-Lumper is the reverse calculation block of the ACRRL Lumper, which was described above. The same pre-processing table of Eq. 18 is created via the cluster analysis in the ACRRL De-Lumper. In ACRRL De-Lumper, the input values are the mole fractions of m thermodynamic lumps: ylump [1 . . . m] and molecular mapping profile: ymap[1 . . . n]. The output is a vector of mole fractions of n molecular species: ymol[1 . . . n] calculated by Eq. 21. Similarly, the equations in the De-Lumper are written in terms of the Aspen EO format including residuals, sparsity patterns and analytical jacobians.

$$ymol [ {SpcIndex}_{i j} ] = ylump [ i ] . ymap [ {SpcIndex}_{i j} ] \\ when {SpcIndex}_{i j} \geq 0 \qquad Eq . 21$$

ACRRL may be applied to selected VLE and LLE cases as described here. The most common process unit ops in refineries and chemicals are VLE based separations such as distillation columns and flash separators. To leverage in the order of 10,000 molecules with those VLE separations via ACRRL, physical properties need to be determined as input criteria of ACRRL. This can be first approached with the calculation of Kᵢ.

The governing equation of VLE in thermodynamics is Eq. 22.

$${\hat{{f}_{i}}}^{L} = {\hat{{f}_{i}}}^{V} \qquad Eq . 22$$

\hat{f}ᵢ^{V} is the fugacity of component I in vapor phase.
\hat{f}ᵢ^{L} is the fugacity of component I in liquid phase.

For non-ideal solutions, there are two typical approaches to address Eq. 22: activity coefficient method and Equation of State (EOS) method.

Activity coefficient method uses an activity coefficient model to address \hat{f}ᵢ^{L} shown in Eq. 23

$${\hat{{f}_{i}}}^{L} = {γ}_{i} \cdot {x}_{i} \cdot {f}_{i}^{0} \qquad Eq . 23$$

fᵢ⁰ is the fugacity of pure component i. At moderate conditions, if the Poynting factor is ignored, fᵢ⁰ is simplified to the saturate vapor pressure of component i: Pᵢ^{Sat}
\hat{f}ᵢ^{V} can be expressed as Eq. 24

$${\hat{{f}_{i}}}^{V} = {ϕ}_{i}^{V} \cdot {y}_{i} \cdot P \qquad Eq . 24$$

ϕᵢ^{V} is the fugacity coefficient of component i in the vapor phase
P is the pressure of the system

At moderate conditions, ϕᵢ^{V} is close to 1, so the equation to estimate Kᵢ is shown as Eq. 25.

$${K}_{i} = \frac{{γ}_{i} \cdot {P}_{i}^{s a t}}{P} \qquad Eq . 25$$

γᵢ can be estimated by an activity coefficient model.

The EOS method uses Eq. 4 for both vapor and liquid phases and thus estimates Kᵢ as Eq. 26.

$${K}_{i} = \frac{{ϕ}_{i}^{L}}{{ϕ}_{i}^{V}} \qquad Eq . 26$$

ϕᵢ^{V} and ϕᵢ^{L} can both be solved via EOS.

Aspen Properties provides a large number of thermodynamics models to address Eq. 25 and Eq. 26 for different systems to calculate Kᵢ in typical VLE blocks (e.g., flash units, columns, etc.).

By analyzing Eq. 22 to Eq. 26, it is observed that Pᵢ^{Sat} is the important apparent property for determining Kᵢ. In addition, hydrocarbon mixtures in refining processes can be approximated as ideal solutions (set γᵢ=1). So Raoult's law is a sufficient approximation for hydrocarbon mixtures at moderate conditions.

$${K}_{i} = \frac{{P}_{i}^{s a t}}{P} \qquad Eq . 27$$

So, Pᵢ^{Sat} is one choice of criterion to use in RRL and has been verified in flash calculations by the Klein Research Group (KRG) and China Petroleum University (CUP). In some embodiments, where Pᵢ^{Sat} is not a direct input property but may be obtained from sophisticated correlation models or EOS models. In addition, in some embodiments the assay-based hypo components cannot directly be defined by Pᵢ^{Sat}. So Pᵢ^{Sat} cannot be used to design a direct lumping/de-lumping algorithm between molecular species in certain embodiments. Therefore, alternative criterion compatible with those embodiments are described. From the nature of phase change, the heat of evaporation and the entropy of evaporation are the fundamental specs in VLE. The corresponding apparent properties in terms of temperature and pressure are boiling point (Tb) and saturate vapor pressure (Pᵢ^{Sat}). Tb is an alternative choice of the criterion for RRL that may be considered. The relationship between Tb and Pᵢ^{Sat} at can be shown in the Clausius-Clapeyron equation.

$$ln \frac{{P}_{i}^{s a t}}{{P}_{0}} = \frac{Δ {H}_{{vap}_{i}}}{R \cdot {T}_{{b}_{i}}} ( \frac{T - {T}_{{b}_{i}}}{T} ) \qquad Eq . 28$$

In Eq. 28, T_{b}_{ᵢ} is the normal boiling point (NBP) of a component i. P₀, the reference pressure of T_{b}_{ᵢ}, is 1 atm. R is the ideal gas constant. ΔHᵥₐₚ_{ᵢ} is the heat of evaporation of component i. T is the temperature of the system. For hydrocarbon components in refining, the temperature effects to ΔHᵥₐₚ_{ᵢ} can be ignored and treated as a constant variable. So Pᵢ^{Sat} of a component i is a function of T_{b}_{ᵢ} and ΔHᵥₐₚ_{ᵢ} at a given condition.

In Eq. 28, ΔHᵥₐₚ_{ᵢ} and T_{b}_{ᵢ} can be approximately expressed as the entropy of evaporation ΔS̅ᵥ̅ₐ̅ₚ̅_̅{̅ᵢ̅}̅:

$$Δ \overline{{S}_{{vap}_{i}}} = \frac{Δ {H}_{{vap}_{i}}}{{T}_{{b}_{i}}} \qquad Eq . 29$$

The Trouton Rule as described in Trouton, F., Nature, 27,292 (1883) gives an approximately value of ΔS̅ᵥ̅ₐ̅ₚ̅ for most liquid components as Eq. 30.

$$Δ \overline{{S}_{v a p}} \approx 10.59 R \qquad Eq . 30$$

Eq. 30 is a good approximation for hydrocarbon mixtures in refining. Therefore Eq. 28 can be simplified to Eq. 31

$$ln \frac{{P}_{i}^{s a t}}{{P}_{0}} \approx 10.59 ( \frac{T - {T}_{{b}_{i}}}{T} ) \qquad Eq . 31$$

In Eq. 31, the normal boiling point (NBP) of a component I, T_{b}_{ᵢ}, is directly related to the saturate vapor pressure Pᵢ^{Sat}. Therefore, T_{b}_{ᵢ} is an alternate criterion for RRL instead of Pᵢ^{Sat}. Moreover, the normal boiling point is the one of the properties used to define thermodynamic lumps as assay hypos in Aspen HYSYS and Aspen Plus. So T_{b}_{ᵢ} is the optimal criterion of RRL that may be used to be compatible with Aspen HYSYS and Aspen Plus.

As a result, boiling point is selected as the criterion to model VLE separation units in Aspen HYSYS and Aspen Plus. To verify this approach, a flash is selected as the VLE block to test. The example is a High-Pressure Separator (HPS) 210 of a MB HCR reactor shown in FIG. 1. To illustrate the concept of this approach, it is assumed the MB stream is an ideal solution and thus apply the Rachford-Rice method to create a MB basic flash block that has a built-in ACRRL function. Eq. 27 is the estimation function of Kᵢ in this MB basic flash. Notice it does not mean the VLE model used in ACRRL approach needs to be simplified to the MB basic flash. The HPS flash example that was selected includes 1366 molecules. A MB flash model using all of the molecules is called MB full VLE flash model, which serves as the reference case to compare. The MB flash model using thermodynamic lumps is called MB lumped VLE flash model. As mentioned above, ACRRL allows users to specify a portion of small isomers without lumping. For this HPS flash, the cluster analysis in ACRRL is used to lump all molecules having the carbon number >=5 and keep other small molecules individually. Then, 1366 molecules are transferred to 84 VLE thermodynamic lumps as the inlet stream in the MB lumped flash model and then reverse those lumps back to 1366 molecules in the products of vapor phase and liquid phase respectively.

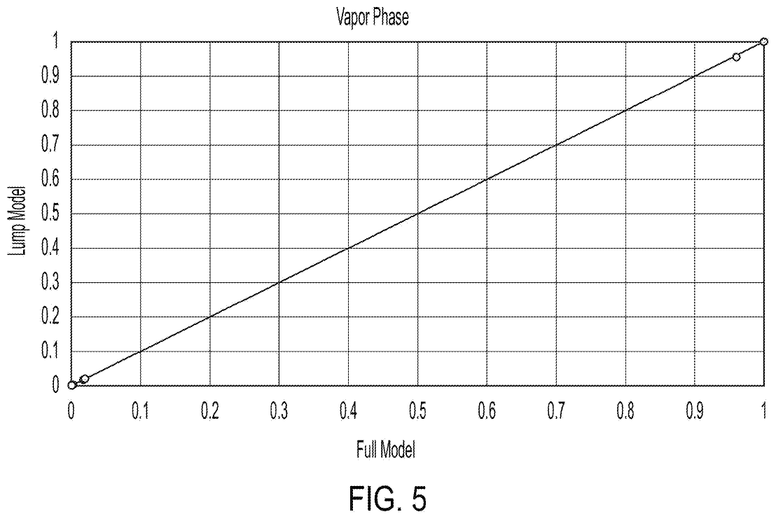

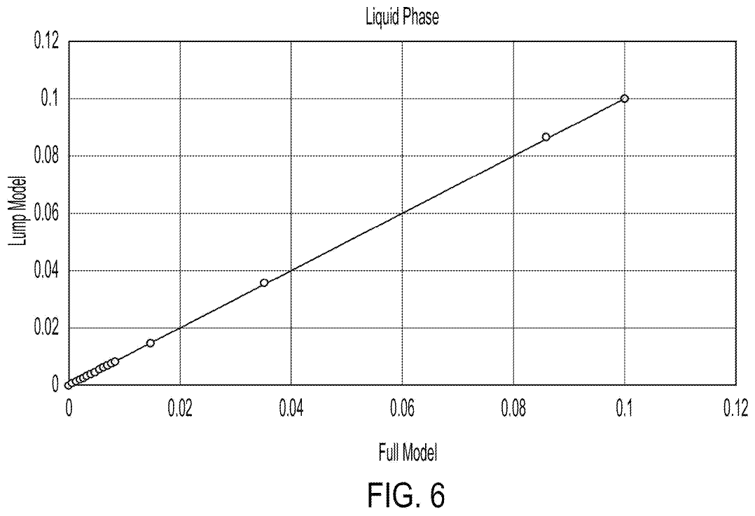

FIGS. 5 and 6 are parity plots that illustrate the comparison of results of the process. The points in the x axis of FIGS. 5 and 6 are the mole fractions of 1366 molecular compositions in the vapor phase and liquid phase of the flash product estimated by the MB full flash model. The points in the y axis of FIGS. 5 and 6 are the mole fractions of 1366 molecular compositions in the vapor phase and liquid phase of the flash product mapped back by ACRRL from the 84 hypo MB lumped flash model. The conditions of this flash test are temperature=65 C and pressure=10 MPa which are typical HPS conditions in a HCR flowsheet. FIGS. 5 and 6 show very good agreement of the distributions of molecular compositions both in vapor phase and liquid phase between the results directly estimated from the MB full flash model and the results mapped via ACRRL from 84 hypos MB reduce model.

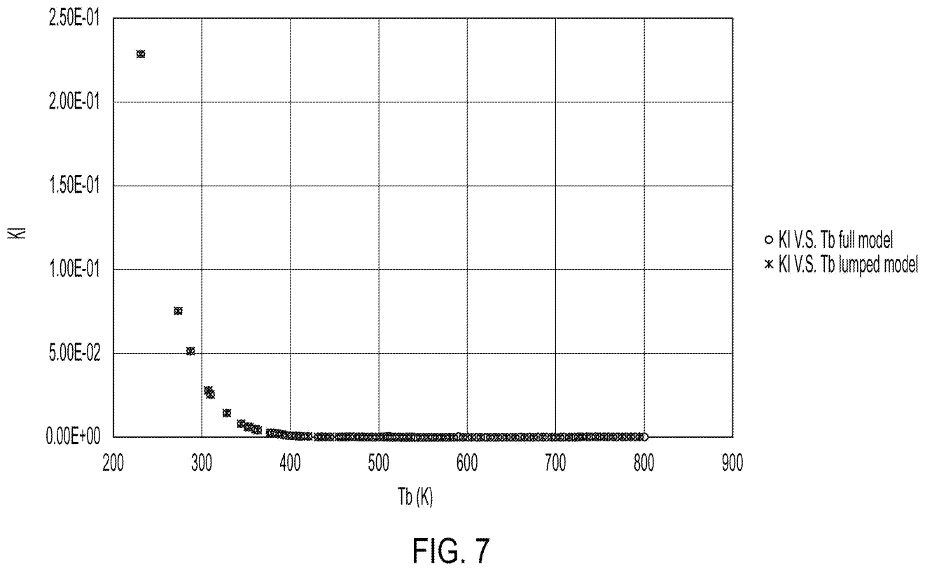

In addition, FIG. 7 shows the curves of vapor-liquid distribution ratio (Kᵢ) values versus boiling point for MB full flash model and MB lumped flash model. From FIG. 7, the distribution curve of Kᵢ and boiling point (Tb) for the MB full flash model is very close to that of the MB lumped flash model. Therefore, Tb is verified to be an optimal alternate criterion of Kᵢ for VLE models.

The test result of the HPS flash in a MB HCR flowsheet shows ACRRL works well in the VLE flash blocks of refining processes. As an extension, ACRRL is not limited to the basic flash in the above test, flash blocks with comprehensive VLE models are also applicable for that approach. The column is one important unit operation in refining processes. For example, there are two kinds of column unit blocks in the flowsheet of Aspen HPR: short cut distillation (SCD) and rigorous distillation column. The essential theory of SCD is summarized by Eq. 29 to Eq. 31, so this approach is inherently applicable for SCD. A rigorous distillation column requires complicated VLE calculations for each tray. The bulk properties (e.g., Molecular Weight (MW), density) of VLE lumps in Aspen HYSYS columns may need to be updated when the mole fraction profiles of molecular compositions are changed. However, the fundamental assumption of ACRRL is that the molecular compositions of refining hydrocarbon mixtures in each VLE lump defined by RRL have the same Kᵢ as shown Eq. 22, Eq. 26 and Eq. 27, which is independent of the properties (e.g., MW, density, criterial properties, binary coefficients, acentric factor, etc.) required to be evaluated in order to solve Eq. 26 and Eq. 27 via EOS and activity coefficient models. In addition, the ideal solution approximation of the hydrocarbon compositions is well verified for industrial purposes. So using ACRRL to model a rigorous distillation column may not be quite as accurate as modeling a flash unit or a SCD, but it is still an accurate approximation for industrial refining processes. The boiling-point-based ACRRL can still obtain good approximate results for both yields and product properties in a Crude Distillation Unit (CDU) model.

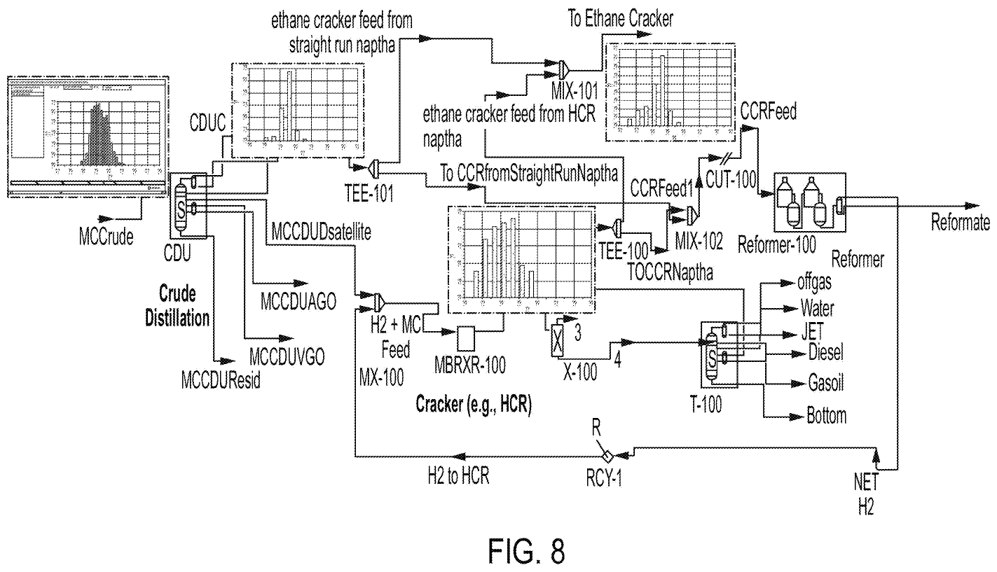

Therefore, boiling point may be used as the criterion and apply ACRRL to all VLE separations involved in Aspen HPR: flash, SCD and rigorous columns, etc. As a result, the molecular compositions of MB models can be reversibly connected with assay-based hypos and can be propagated through a large Aspen HPR flowsheet shown in FIG. 8.

In addition to VLE separation problems, LLE based extraction processes also play very important roles in hydrocarbon upgrading processes especially for Crude to Chemical (CTOC) situations because the extraction is the main separation technique to perform separation processes for heavy resid or asphaltene, which accounts for a large portion of crudes. The extraction process of heavy resid is not just a standalone unit op such as deasphaltene extractor, but typically works in tandem with reactors such as resid FCC, resid hydroprocessing, etc. It is a challenge for conventional flowsheet software to propagate compositions of heavy hydrocarbon mixtures across extractors in a refining flowsheet because most of components in the software are defined by boiling points which is not applicable for LLE extraction. However, molecular models can be used and ACRRL can be used to address it. The inlet stream of a given extraction process can be either a portion of crude oil or a product stream from a reactor. Molecular characterization (MC) may be used to calculate the molecular composition of the crude or the relevant portion of it and estimate the molecular composition of the product stream of a conversion unit via MB reactor. As a result, molecular composition of the inlet stream of that extraction process can be described. Selecting appropriate LLE criteria, ACRRL may be applied to transfer the molecular compositions of the inlet stream to a set of LLE thermodynamic lumps. As a result, the LLE model can be calculated in terms of those LLE lumps. The LLE thermodynamic lumps in the products can be reversibly mapped back to molecular compositions and propagated to downstream units. The key point to use this logic is to determine the criteria of LLE. To simplify the problem, the LLE model of heavy oil based on the activity coefficient model and regular solution theory can be analyzed. The governing equation of LLE is shown in Eq. 32 and the Kᵢ of a hydrocarbon molecule in different liquid phases is written as a simplified expression in Eq. 33:

$${\hat{{f}_{i}}}^{{L}_{1}} = {\hat{{f}_{i}}}^{{L}_{2}} \qquad Eq . 32$$

\hat{f}ᵢ^{L}^{₁} is the fugacity of component i in light liquid phase.
\hat{f}ᵢ^{L}^{₂} is the fugacity of component i in heavy liquid phase.

$${K}_{i} = \frac{{x}_{i 2}}{{x}_{i 1}} = \frac{{γ}_{i 1}}{{γ}_{i 2}} \qquad Eq . 33$$

γᵢ₁ and γᵢ₂ are activity coefficients of component i in the light liquid phase and the heavy liquid phase.
xᵢ₁ and xᵢ₂ are the mole fractions of component i in the light liquid phase and the heavy liquid phase.
Kᵢ is the distribution ratio of component i in the heavy liquid phase and the light liquid phase.

The Flory-Huggins solution theory can be applied to estimate active coefficients in a given phase for heavy hydrocarbon mixtures as shown in Eq. 34

$$ln {γ}_{i} = 1 + ln ( \frac{{V}_{i}}{\overline{V}} ) - \frac{{V}_{i}}{\overline{V}} + \frac{{V}_{i}}{R \cdot T} {( {δ}_{i} - \overline{δ} )}^{2} \qquad Eq . 34$$

γᵢ is the activity coefficient of component i in a given phase
Vᵢ is the molar volume of component i in a given phase
δᵢ is the solubility parameter of component i in a given phase
V̅ is the average molar volume of all components in a given phase
δ̅ is the volumetric average solubility parameter of all components in a given phase

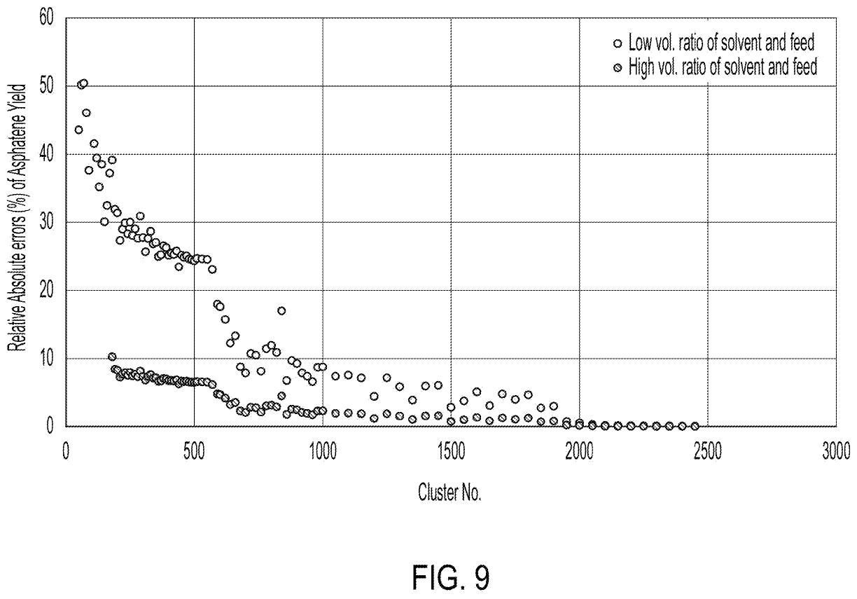

From Eq. 34, Vᵢ and δᵢ are two properties to estimate Kᵢ and thus can be used as the criteria in ACRRL for hydrocarbon LLE models. To validate this approach, a heavy asphaltene precipitation process was selected to simulate. The asphaltene precipitation can be described as a LLE flash process. The solute is a heavy oil with high asphaltene content. The solvent is a combination of a poor solvent (n-heptane or n-pentane) and a good solvent (toluene). By simulating a set of mixing ratios, an asphaltene precipitation curve can be calculated. The first task is to figure out an optimal cluster number for ACRRL for that asphaltene LLE model. In this example, the inlet asphaltene stream has ˜3000 molecules. The number of clusters was set from 50 to 3000 in ACRRL to simulate the flash calculation. The MB LLE flash based on Eq. 34 is used. The results of modeling the extraction of a mixture of the inlet heavy oil stream and n-heptane via MB LLE flash in terms of different lumps are shown in FIG. 9.

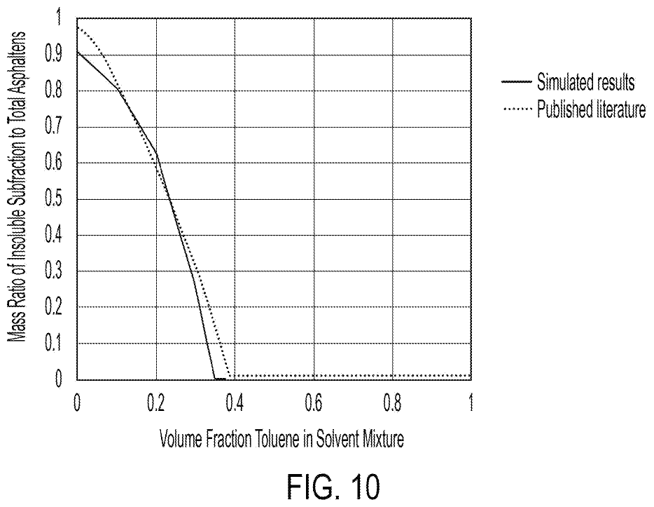

In FIG. 9, the y axis is the relative absolute error (%) of asphaltene precipitation yield between the results with the specified number of clusters and the results without any lumping. The x axis is the number of clusters used in ACRRL. There are two curves in the plot representing different conditions for the ratio of the solvent and the heavy oil sample. One set is at the low volume ratio and the other set is at the high volume ratio. The relative difference in the results is <10% when the cluster number is ˜100 if the case is simulated under the condition of a higher volume ratio between solvent and feed (case 1) and the relative difference in the results is <10% when the cluster number is ˜700 if the case is simulated under the condition of a lower volume ratio between solvent and feed (case 2). Case 2 requires more lumps than case 1 to reach a similar accuracy because the derivative of the precipitation curve of case2 is much larger than the derivative of the precipitation curve of case1. The number of lumps used in ACRRL is dependent on the purpose and operating conditions of the model, but it is significantly reduced in both cases and thus optimally allows maintaining affordable computational resources and acceptable accuracy for the case. Next, the configuration of 100 LLE lumps in ACRRL is used to simulate the asphaltene precipitation curve by changing a set of solvent mixing ratios of n-heptane and toluene. The total volume ratio between solvent and asphaltene is kept at a ratio of 40:1. FIG. 10 compares the simulated results (solid line) with results from published literature (dotted line) (Mannistu et al., Solubility Modeling of Asphaltenes in Organic Solvents, Energy Fuels 1997, 11, 3, 615-622 (1997)). The simulated results from MB LLE flash show good agreement with the curve in the paper and thus validates the approach in the embodiments: Vᵢ and δᵢ can be used as optimal criteria in ACRRL to model LLE processes.

Even in situations where the MB basic LLE flash based on Eq. 34 may not work as well, for example with mixtures having strong polar components, it does not affect the application of the ACRRL approach to general extraction processes. Users can use a more complex LLE model (e.g., NRTL models, EOS, PC-SAFT, etc.) for such a system and the core part of ACRRL is to provide appropriate thermodynamics LLE lumps to that comprehensive model in a flexible way. The analysis of LLE criteria of ACRRL is still sufficient to that system and users always can update the property (e.g., Hansen solubility parameters to account the polarity) to improve the lumping/delumping of ACRRL.

In summary, ACRRL allows for the reduction in the number of components from the MB model used in separation blocks while maintaining the full molecular detail. The criterion of ACRRL is flexible to configure for different separation processes (e.g, VLE, LLE). ACRRL provides the user a flexible option to control the size and granularity of the model by cluster analysis. The molecular compositions can be reversibly mapped back after the separation calculation. The results from ACRRL have been validated for a VLE flash test and a LLE flash test. In general, ACRRL is not limited to VLE and LLE processes. By selecting appropriate criteria (e.g., melting point, enthalpy of fusion (ΔH_{fus})), this technique may also apply to solid-liquid separation processes. Driven by first-principles knowledge of any process chemistries, ACRRL can reduce the number of numerical variables to an acceptable number for simulation by capturing the similarity of molecules in nature while maintaining the full details of molecular compositions. A flowsheet that can propagate the molecular compositions across wide range process models has been addressed and multi-unit simulation of CTOC cases can be modeled at the molecular level.

### Computer Implementation

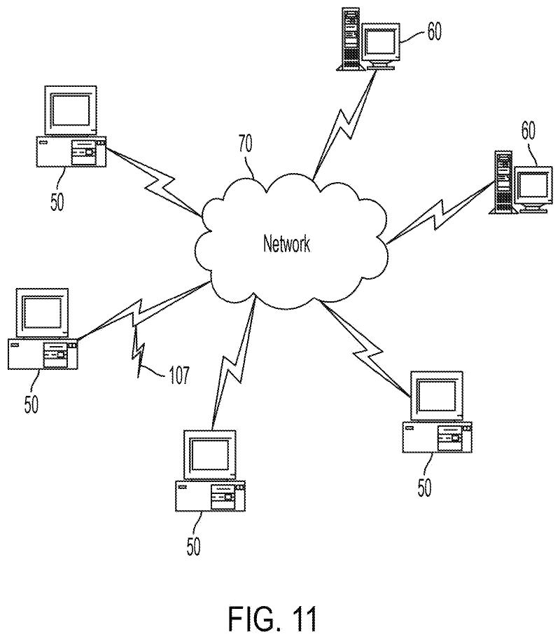

FIG. 11 illustrates a computer network or similar digital processing environment in which the present invention may be implemented.

Client computer(s)/devices 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable.

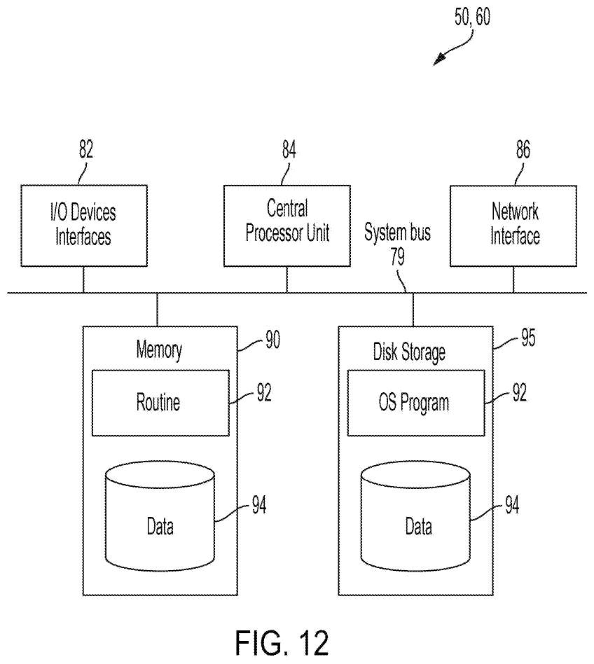

FIG. 12 is a diagram of the internal structure of a computer (e.g., client processor/device 50 or server computers 60) in the computer system of FIG. 11. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 11). Memory 90 provides volatile storage for computer software instructions 92 and data 94 (such as method 220, MB EORXR, etc. detailed above) used to implement an embodiment of the present invention. Disk storage 95 provides non-volatile storage for computer software instructions 92 and data 94 used to implement an embodiment of the present invention. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, flash drive etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

In other embodiments, the program product 92 may be implemented as a so called Software as a Service (SaaS), or other installation or communication supporting end-users.

Embodiments or aspects thereof may be implemented in the form of hardware, firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and thus, the data processors described herein are intended for purposes of illustration only and not as a limitation of the embodiments.

### Incorporation by Reference; Equivalents

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A computer-implemented method for controlling a separation process, the method comprising, in a computer:
representing a feedstock of the chemical separator as a collection of molecules, each molecule having a mole fraction;
performing a cluster analysis on a property of molecules of the collection of molecules to generate thermodynamic lumps;
generating a mapping identity table that identifies each molecule of the collection of molecules in the feedstock;
performing a simulation of a chemical separation of the thermodynamic lumps to generate a mole fraction of each thermodynamic lump in a resultant first phase and a mole fraction of each thermodynamic lump in a resultant second phase;
determining the mole fraction of molecules in the resultant first phase and the mole fraction of molecules in the resultant second phase based on the mapping identity table, the mole fraction of each thermodynamic lump in the resultant first phase, and the mole fraction of each thermodynamic lump in the resultant second phase; and
controlling a separation process based on one or more of the mole fraction of molecules in the resultant first phase and the mole fraction of molecules in the resultant second phase.

2. The method of claim 1, wherein the property is a thermodynamic property of molecules of the collection of molecules.

3. The method of claim 2, wherein the thermodynamic property is a Kᵢ criteria.

4. The method of claim 3, wherein the Kᵢ criteria is boiling point.

5. The method of claim 3, wherein the Kᵢ criteria is vapor pressure.

6. The method of claim 3, wherein the Kᵢ criteria is a solubility parameter.

7. The method of claim 3, wherein the Kᵢ criteria is melting point.

8. The method of claim 3, wherein the Kᵢ criteria is enthalpy of fusion.

9. The method of claim 1, wherein the property is a structural attribute of molecules of the collection of molecules.

10. The method of claim 9, wherein the structural attribute comprises compound class.

11. The method of claim 10, wherein the compound class comprises one or more of aromatic, naphthenic, isoparaffin, paraffin, and olefin.

12. The method of claim 9, wherein the structural attribute comprises number of carbon atoms.

13. The method of claim 9, wherein the structural attribute comprises compound class and number of carbon atoms.

14. The method of claim 1, wherein the cluster analysis is K-Mean method AS136.

15. The method of claim 1, wherein the cluster analysis is K-Mean method AS58.

16. The method of claim 1, wherein the cluster analysis is Ward's minimum variance method.

17. The method of claim 1, further comprising receiving user input selecting the cluster analysis.

18. The method of claim 1, further comprising receiving user input selecting a total number of thermodynamic lumps.

19. The method of claim 1, further comprising receiving user input selecting a maximum number of molecular species in the thermodynamic lumps.

20. The method of claim 1, further comprising receiving user input selecting particular molecules from the collection of molecules for a thermodynamic lump.

21. The method of claim 1, further comprising receiving user input selecting particular molecules from the collection of molecules that are excluded from a thermodynamic lump.

22. The method of claim 1, wherein the resultant first phase is a vapor phase and the resultant second phase is a liquid phase.

23. The method of claim 1, wherein the resultant first phase is a liquid phase and the resultant second phase is a liquid phase.

24. The method of claim 1, wherein the resultant first phase is a solid phase and the resultant second phase is a liquid phase.

25. (canceled)

26. A system for controlling a separation process, the system comprising:
a processor; and
a memory with computer code instructions stored thereon, the processor and the memory, with the computer code instructions, being configured to cause the system to:
represent a feedstock of the chemical separator as a collection of molecules, each molecule having a mole fraction;
perform a cluster analysis on a property of molecules of the collection of molecules to generate thermodynamic lumps;
generate a mapping identity table that identifies each molecule of the collection of molecules in the feedstock;
perform a simulation of a chemical separation of the thermodynamic lumps to generate a mole fraction of each thermodynamic lump in a resultant first phase and a mole fraction of each thermodynamic lump in a resultant second phase;
determine the mole fraction of molecules in the resultant first phase and the mole fraction of molecules in the resultant second phase based on the mapping identity table, the mole fraction of each thermodynamic lump in the resultant first phase, and the mole fraction of each thermodynamic lump in the resultant second phase; and
control a separation process based on one or more of the mole fraction of molecules in the resultant first phase and the mole fraction of molecules in the resultant second phase.

27-50. (canceled)
